(19) United States
(12) Patent Application Publication
(10) Pub. No.: US 2007/0238878 A1
(43) Pub. Date: Oct. 11, 2007

(54) PROCESS FOR THE SYNTHESIS OF 4-(3-SULFONYLPHENYL)-PIPERIDINES

(76) Inventors: Richard Desmond, Lebanon, NJ (US); Paul N. Devine, Tinton Falls, NJ (US); Clas Sonesson, Billdal (SE); Donald R. Gauthier, Westfield, NJ (US)

Correspondence Address: GAUTHIER & CONNORS, LLP, 225 FRANKLIN STREET, SUITE 2300, BOSTON, MA, 02110, US

(21) Appl. No.: 11/734,977

(22) Filed: Apr. 13, 2007

Related U.S. Application Data
(63) Continuation of application No. PCT/EP05/11021, filed on Oct. 13, 2005.
(60) Provisional application No. 60/618,194, filed on Oct. 13, 2004.

(51) Int. Cl. C07D 211/18

(52) U.S. Cl. 546/232

(57) ABSTRACT

The present invention is directed to processes for the preparation of 4-(sulfonylphenyl)-piperidines of the Formula (VI), and pharmaceutically acceptable salts thereof; which comprises oxidizing a sulfide of the Formula (VII) or Formula (VIII), to give a compound of the Formula (IX) or Formula (X) respectively, followed by catalic reduction of the compound of the Formula (IX) or dehydration of compound of formula (X) to give a compound of Formula (IX) followed by catalytic reduction of the compound formula (IX) to give the compound of the Formula (VI).

### BACKGROUND OF THE INVENTION

4-(3-Methanesulfonylphenyl)-1-N-propylpiperidine is useful as a modulator of dopamine neurotransmission and has therapeutic application for example in the treatment of Alzheimer's disease, Parkinson's disease and schizophrenia. Synthetic methods to prepare 4-(sulfonylphenyl)piperidines have been described in PCT Patent Publications WO 01/46145 and WO 01/46145.

In accordance with the present invention, processes are provided for the preparation of 4-(sulfonylphenyl)piperidines, and pharmaceutically acceptable salts thereof. The subject process provide 4-(sulfonylphenyl)piperidines in high yield and purity while minimizing the number of synthetic steps.

### SUMMARY OF THE INVENTION

The present invention is directed to processes for the preparation of 4-(sulfonylphenyl)-piperidines of the formula VI: 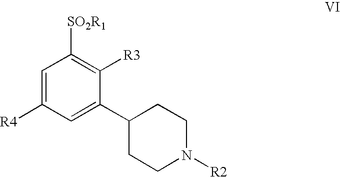
wherein:
R¹ is selected from the group consisting of:

(1) —CH₃, and

(2) —CH₂CH₃;

R² is selected from the group consisting of:

(1) —CH₂CH₃,

(2) —CH₂CH₂CH₃,

(3) —CH₂CH₂CH₂CH₃,

(4) —CH(CH₃)CH₂CH₃,

(5) —CH₂CH(CH₃)₂,

(6) —CH₂CH₂OCH₃, and

(7) —CH₂CH═CH₂;

R³ is selected from the group consisting of:

(1) hydrogen, and

(2) fluoro;

R⁴ is selected from the group consisting of:

(1) hydrogen, and

(2) fluoro;

and pharmaceutically acceptable salts thereof.

### DETAILED DESCRIPTION OF THE INVENTION

The present invention relates to process for the preparation of 4-(sulfonylphenyl)-piperidines which are useful as pharmaceutical agents.

An embodiment of the present invention is directed to a process for the preparation of a 4-(sulfonylphenyl)-piperidine of the formula VIII: 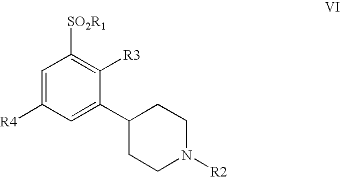
wherein:
R¹ is selected from the group consisting of:

(1) —CH₃, and

(2) —CH₂CH₃;

R² is selected from the group consisting of:

(1) —CH₂CH₃,

(2) —CH₂CH₂CH₃,

(3) —CH₂CH₂CH₂CH₃,

(4) —CH(CH₃)CH₂CH₃,

(5) —CH₂CH(CH₃)₂,

(6) —CH₂CH₂OCH₃, and

(7) —CH₂CH═CH₂;

R³ is selected from the group consisting of:

(1) hydrogen, and

(2) fluoro;

R⁴ is selected from the group consisting of:

(1) hydrogen, and

(2) fluoro;

and pharmaceutically acceptable salts thereof;

which comprises oxidizing a sulfide of the formula VIII or VIII: 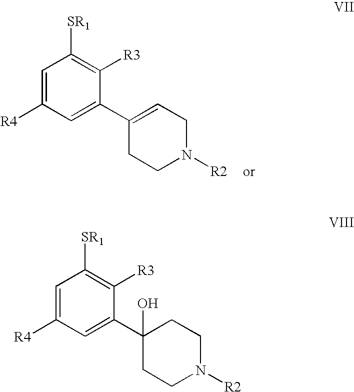
to give a compound of the formula IX or X, respectively: 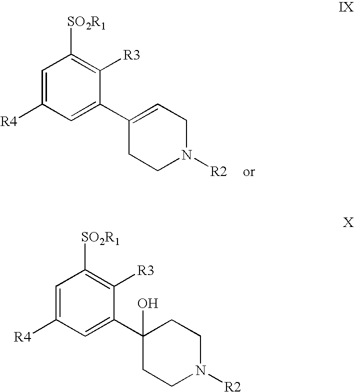
followed by catalytic reduction of the compound of the formula IX or dehydration of compound of formula X to give a compound of formula IX followed by catalytic reduction of the compound of formula IX to give the compound of the formula VI: 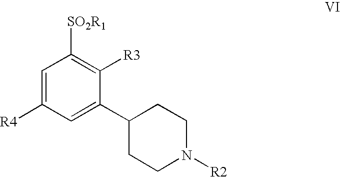
or a pharmaceutically acceptable salt thereof.

The present invention also relates to the above routes individually. In a preferred embodiment of the invention, R³ and R⁴ are not H when R¹=Me and R²=n-Pr.

An embodiment of the present invention is directed to a process for the preparation of 1-ethyl-4-[2-fluoro-3-(methylsulfonyl)phenyl]piperidine of the formula I: 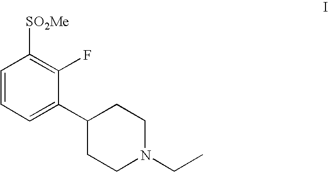
or a pharmaceutically acceptable salt thereof, which comprises oxidizing a sulfide of the formula II: 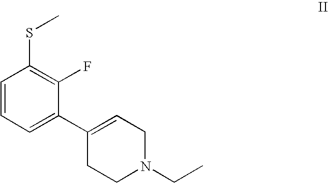
to give a compound of the formula III: 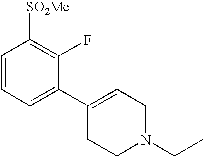
followed by catalytic reduction of the compound of the formula III to give the compound of the formula I: 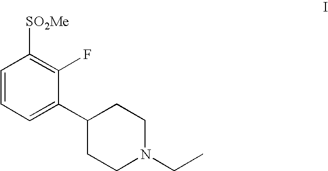
or a pharmaceutically acceptable salt thereof.

A further embodiment of the present invention is directed to a process for the preparation of 1-ethyl-4-[2-fluoro-3-(methylsulfonyl)phenyl]piperidine of the formula I: 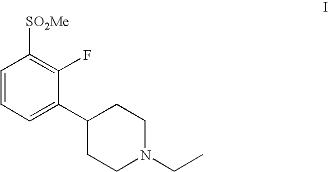
or a pharmaceutically acceptable salt thereof, which comprises oxidizing a sulfide of the formula IV 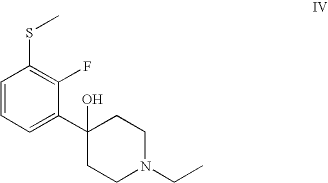
to give a compound of the formula V: 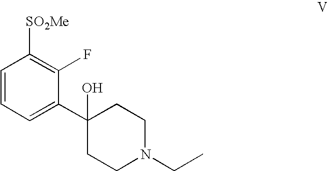
followed by dehydration of the compound of the formula V with strong acid; to give the compound of the formula III: 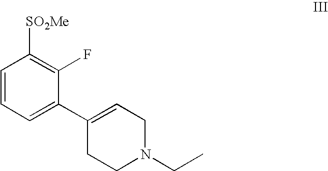
followed by catalytic reduction of the compound of the formula III; to give the compound of the formula I: 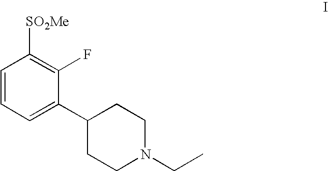
or a pharmaceutically acceptable salt thereof.

A further embodiment of the present invention is directed to a process for the preparation of 1-ethyl-4-[2-fluoro-3-(methylsulfonyl)phenyl]piperidine of the formula I: 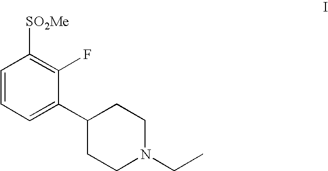
or a pharmaceutically acceptable salt thereof, which further comprises; dehydrating an alcohol of the formula IV: 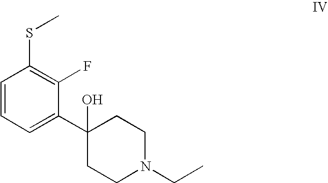
with a strong acid; to give a sulfide of the formula II: 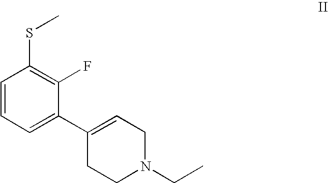
oxidizing the sulfide of the formula III to give a compound of the formula III: 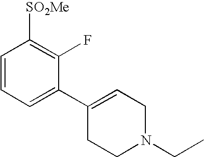
followed by catalytic reduction of the compound of the formula III; to give the compound of the formula I: 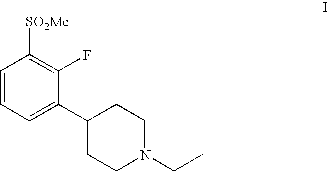
or a pharmaceutically acceptable salt thereof.

A further embodiment of the present invention is directed to a process for the preparation of 1-ethyl-4-[3-fluoro-5-(methylsulfonyl)phenyl]piperidine of the formula XI: 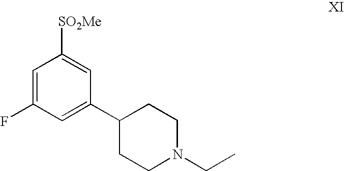
or a pharmaceutically acceptable salt thereof, which comprises oxidizing a sulfide of the formula XII: 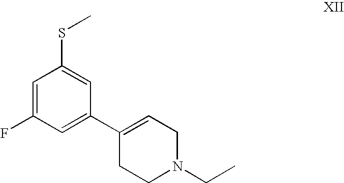
to give a compound of the formula XIII: 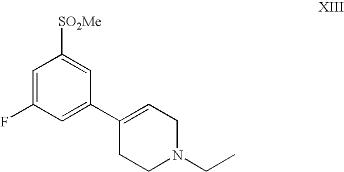
followed by catalytic reduction of the compound of the formula XIII, to give the compound of the formula XI: 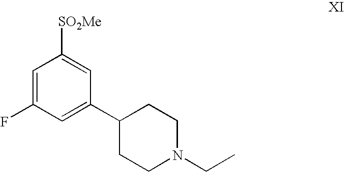
or a pharmaceutically acceptable salt thereof.

A further embodiment of the present invention is directed to a process for the preparation of 1-ethyl-4-[3-fluoro-5-(methylsulfonyl)phenyl]piperidine of the formula XI: 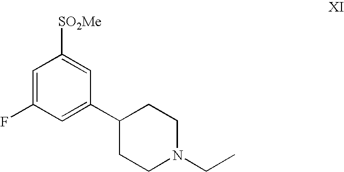
or a pharmaceutically acceptable salt thereof, which further comprises dehydrating an alcohol of the formula XIV: 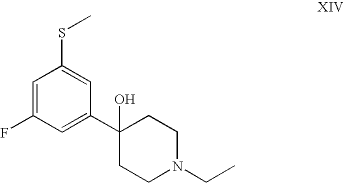
with a strong acid; to give a sulfide of the formula XII: 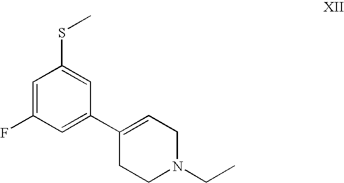
followed by oxidizing the sulfide of the formula XII to give a compound of the formula XIII: 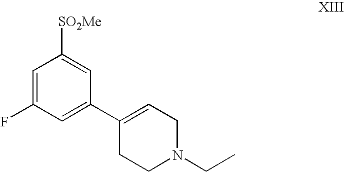
followed by catalytic reduction of the compound of the formula XIII to give the compound of the formula XI: 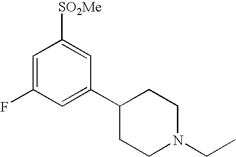
or a pharmaceutically acceptable salt thereof.

A further embodiment of the present invention is directed to a process for the preparation of 1-ethyl-4-[3-fluoro-5-(methylsulfonyl)phenyl]piperidine of the formula XI: 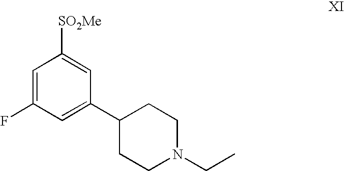
or a pharmaceutically acceptable salt thereof, which further comprises oxidizing the sulfide of the formula XIV 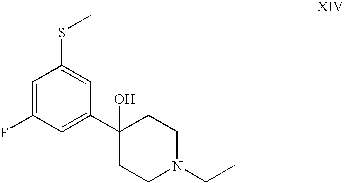
to give a compound of the formula XV: 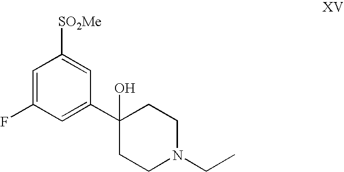
followed by dehydrating of the compound of the formula XV with a strong acid to give a compound of the formula XIII: 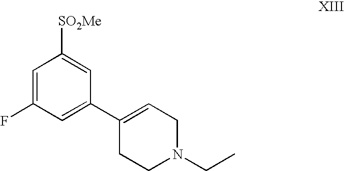
followed by catalytic reduction of the compound of the formula XIII to give the compound of the formula XI: 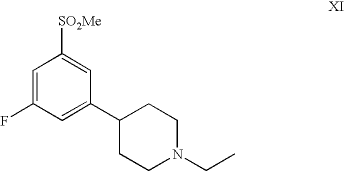
or a pharmaceutically acceptable salt thereof.

In an embodiment of the present invention the strong acid is a strong inorganic acid or a strong organic acid. In an embodiment of the present invention the strong acid is selected from sulfuric acid, hydrochloric acid, hydrofluoric acid, phosphoric acid, polyphosphoric acid nitric acid and trifluoroacetic acid. Optionally, the dehydration of the alcohols of the formulae VIII, IV, XIV, X, V or XV with a strong acid is conducted neat or in a solvent. In an embodiment of the present invention the solvent is selected from toluene, xylene, hexanes and water.

In an embodiment of the present invention, oxidizing a sulfide of the formula VIII, II, XII, IV, XIV or VIII is carried out using a catalytic oxidizing agent, such as a tungsten, ruthenium, rhenium, molybdenum, osmium, silicotungstate (e.g. (Bu₄N)₄[γ-SiW₁₀O₃₄(H₂O)₂]) or chromium oxidizing agent. The addition of imidazole, phosphate, or carboxylates significantly enhances the rate of organic sulfide oxygenation.

In an embodiment of the present invention the catalytic oxidizing agent is a tungsten oxidizing agent. In an aspect of this embodiment, the tungsten oxidizing agent is sodium tungstate.

In an embodiment of the present invention the oxidant is a peroxide. In an aspect of this embodiment, the peroxide is sodium peroxide, hydrogen peroxide, sodium hypochlorite, sodium bromate, sodium periodate, peroxyacetic acid or peroxybenzoic acid. In a further aspect of this embodiment, the peroxide is sodium peroxide. Within this embodiment, the peroxide is an aqueous solution of sodium peroxide.

In another embodiment of the present invention, oxidizing a sulfide of the formula VII, II, XII, IV, XIV or VIII is carried out using a stoichiometric oxidant. Preferred stoichiometric oxidants are peroxides, oxone, MCPBA or KMnO₄. Catalytic oxidizing agents as detailed above are, however, preferable.

In an embodiment of the present invention the step of oxidizing the sulfide of the formula VII, II, XII, IV, XIV or VIII is conducted at less than 3 pH. Within this embodiment, the step of oxidizing the sulfide of the formula VII, II, XII, IV, XIV or VIII is conducted at less than 2 pH. Further within this embodiment, the step of oxidizing the sulfide of the formula VIII, II, XII, IV, XIV or VIII is conducted at less than 1 pH.

In an embodiment of the present invention the step of oxidizing the sulfide of the formula VII, II, XII, IV, XIV or VIII is conducted at a temperature greater than 30° C. (inclusive). Within this embodiment, the step of oxidizing the sulfide of the formula VIII, II, XII, IV, XIV or VIII is conducted at a temperature greater than 40° C. (inclusive). Further within this embodiment, the step of oxidizing the sulfide of the formula VIII, II, XII, IV, XIV or VIII is conducted at a temperature between 40° C. and 60° C. (inclusive). Further within this embodiment, the step of oxidizing the sulfide of the formula VIII, II, XII, IV, XIV or VIII is conducted at a temperature between 50° C. and 55° C. (inclusive).

Preferred solvents for conducting the step of oxidizing the sulfide of the formula VII, II, XII, IV, XIV or VIII comprise an aqueous solution with an organic solvent which is selected from toluene, tetrahydrofuran (THF), diethyl ether, diglyme and methyl t-butyl ether. The most preferred organic solvent is toluene.

In an embodiment of the present invention the step of catalytic reduction of the compound of the formula IX, III or XIII comprises catalytic hydrogenation. Within this embodiment, the step of catalytic reduction of the compound of the formula IX, III or XIII comprises catalytic hydrogenation with a palladium catalyst, a platinum catalyst or a ruthenium catalyst. Within this embodiment, the step of catalytic reduction of the compound of the formula IX, III or XIII comprises catalytic hydrogenation with a palladium catalyst. Within this embodiment, the step of catalytic reduction of the compound of the formula IX, III or XIII comprises catalytic hydrogenation with a palladium on carbon catalyst. Further within this embodiment, the step of catalytic reduction of the compound of the formula IX, if III or XIII comprises catalytic hydrogenation with a 10% palladium on carbon catalyst or a 5% palladium on carbon catalyst.

In an alternate embodiment of the present invention the step of catalytic reduction of the compound of the formula IX, III or XIII comprises catalytic transfer hydrogenation. Within this embodiment, the step of catalytic reduction of the compound of the formula IX, III or XIII comprises catalytic transfer hydrogenation with a rhodium catalyst or a ruthenium catalyst and a hydrogen transfer source. Within this embodiment, the rhodium catalyst may be selected from bis((pentamethylcyclopentadienyl)rhodium chloride) and bis((cyclopentadienyl)rhodium chloride), optionally in the presence of alternate ligands. Within this embodiment, the ruthenium catalyst may be selected from bis((4-isopropyl-toluenyl)ruthenium chloride) and bis((cyclopenta-dienyl)ruthenium chloride), optionally in the presence of alternate ligands. Within this embodiment, the hydrogen transfer source may be an acid or an alcohol, such as formic acid, methanol, ethanol, isopropanol, isobutanol or n-butanol. In this embodiment, a base is optionally present with the hydrogen transfer source. The base may be an inorganic base such as a base selected from potassium or sodium hydroxide, potassium or sodium carbonate, potassium or sodium bicarbonate potassium or sodium alkoxides, and the like. The alkoxides can be derived from lower (C₁-C₅) or higher (>C₆) primary, secondary or tertiary alcohols.

Solvents for conducting the step of catalytic reduction of the compound of the formula IX, III or XIII include an aqueous solution with an alcohol, such as an alcohol selected from methanol, ethanol, isopropanol, isobutanol or n-butanol. Within this embodiment, the alcohol may be methanol.

The term “pharmaceutically acceptable salts” refers to salts prepared from pharmaceutically acceptable non-toxic bases or acids including inorganic or organic bases and inorganic or organic acids. Salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are benzenesulfonic, citric, hydrobromic, hydrochloric, maleic, fumaric, succinic and tartaric acids. It will be understood that, as used herein, references to the compounds of the present invention are meant to also include the pharmaceutically acceptable salts.

The starting materials and reagents for the subject processes are either commercially available or are known in the literature or may be prepared following literature methods described for analogous compounds. The skills required in carrying out the reaction and purification of the resulting reaction products are known to those in the art. Purification procedures include crystallization, distillation, normal phase or reverse phase chromatography.

The following Examples are provided by way of illustration only, and in no way are meant to limit the scope of the invention.

### EXAMPLE 1

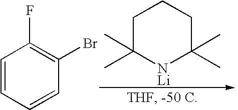

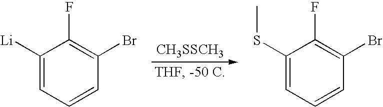

| Materials | Amount | Moles |
|---|---|---|
| 2,2,6,6-tetramethylpiperidine | 0.226 kg | 1.6 eq |
| THF | 0.6 L | |
| Hex-Li (33% in Hexane) | 0.598 L | 1.5 eq |
| 1-bromo-2-fluorobenzene | 0.175 kg | 1.0 eq |
| CH3SSCH3 | 0.133 L | 1.5 eq |
| THF | 0.7 L | |
| Methyl acrylate | 0.180 L | 2.0 eq |
| 2 M NaOH | 1.4 L | 8 eq |
| MTBE | 2 × 1.4 L | |
| 2 M HCl | 2 × 1.05 L | |

To a solution of 2,2,6,6-tetramethylpiperidine in tetrahydrofuran under nitrogen atmosphere at −50° C. was added hexyllithium (−50° C. to −30° C.) The mixture was allowed to warm to ambient temperature and added to a solution of 1-bromo-2-fluorobenzene and dimethyldisulfide in tetrahydrofuran at −78° C. (−80° C. to −60° C.). The mixture was slowly allowed to warm to ambient temperature, cooled to 0° C., and quenched with methylacrylate with the reaction temperature maintained <30° C. throughout the addition followed by the addition of NaOH (2 M). The organic solvent was evaporated and the aqueous phase was extracted with tert-butyl methyl ether. The organic phase washed with an aqueous solution of hydrochloric acid (2 M) and the combined organic phase was evaporated to dryness to give an oil. The residue was purified by column chromatography using 7 mass equivalents of silica compared to the residue and using heptane as the eluent to give the title compound. MS m/z (relative intensity, 70 eV) 222 (M+, bp), 220 (M+, 91), 189 (24), 187 (25), 126 (97).

### EXAMPLE 2

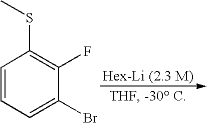

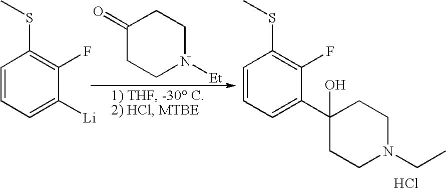

| Materials | Amount | Moles |
|---|---|---|
| 1-bromo-2-fluoro-3-(methylthio)benzene | 11.0 kg | 1.0 eq |
| THF | 55 L | |
| Hex-Li (33% in Hexane) | 14 L | 1.0 eq |
| 1-Ethyl-4-piperidone | 6.012 kg | 0.95 eq |
| THF | 2 L | |
| MeOH | 4 L | |
| 5 M HCl | 44 L | |
| Heptane | 44 L | |
| MTBE | 43 L | |
| 30% NaOH | 33 L | |
| i-PrOAc | 66 L | |
| Sat NaCl | 44 L | |

To a −78° C. solution of 1-bromo-2-fluoro-3-(methylthio)benzene in THF under a nitrogen atmosphere, was added hexyllithium over 50 minutes with the reaction temperature maintained <−60° C. throughout the addition. The mixture was stirred for 1 min at −78° C. and then 1-ethyl-4-piperidone was added over 50 min. with the reaction temperature maintained <−60° C. throughout the addition. The mixture was stirred at −75° C. to −70° C. for 60 min. The reaction mixture was then quenched with MeOH at −70° C. to −60° C. over a period of 9 minutes and then brought to room temperature. The reaction mixture was treated with 5 M HCl at 0-15° C. over a period of 50 minutes. The aqueous mixture was first extracted with heptane and then TBME. The aqueous layer was then basified with 30% NaOH at 0-15° C. over a period of 50 minutes and then extracted with i-PrOAc. The organic layer washed with brine and then evaporated to dryness yielding 11.2 kg of an oily residue. MS m/z (rel. intensity, 70 eV) 269 (M+, 49), 254 (bp), 236 (36), 169 (13), 109 (17).

### EXAMPLE 3

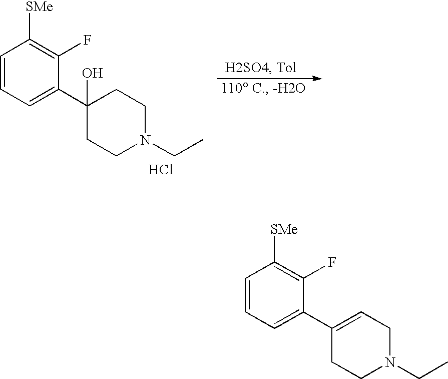

A solution of 1-ethyl-4-[2-fluoro-3-(methylthio)phenyl]piperidin-4-ol (42 g, 156 mmol) and sulfuric acid (18 M, 8.5 ml, 156 mmol) in toluene (200 ml) was refluxed under a Dean-Stark water separator for 15 h. The solution was cooled to ambient temperature, water was added and the phases were separated in a separation funnel. The aqueous phase was cooled to 0° C., made basic with a sodium hydroxide solution (5 M) and extracted with ethyl acetate (2×100 ml). The combined organic phase was dried (MgSO₄) and concentrated to afford the title compound (22.6 g). MS m/z (rel. intensity, 70 eV) 251 (M+, bp), 236 (85), 147 (65), 146 (45), 110 (44).

### EXAMPLE 4

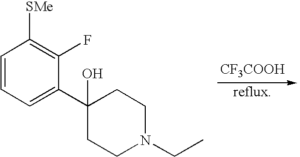

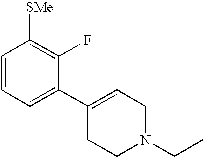

| Materials | Amount | Moles |
|---|---|---|
| 1-ethyl-4-[2-fluoro-3-(methylthio)phenyl]piperidin-4-ol | 11.0 kg | 1.0 eq |
| Trifluoroacetic acid | 42 L | |

Reactor was loaded with 1-ethyl-4-[2-fluoro-3-(methylthio)phenyl]piperidin-4-ol and trifluoroacetic acid and purged with nitrogen (exothermic). The mixture was heated to 82-85° C. for 20 h. The solution was then cooled to room temperature. MS m/z (rel. intensity, 70 eV) 251 (M+, bp), 236 (85), 147 (65), 146 (45), 110 (44).

### EXAMPLE 5

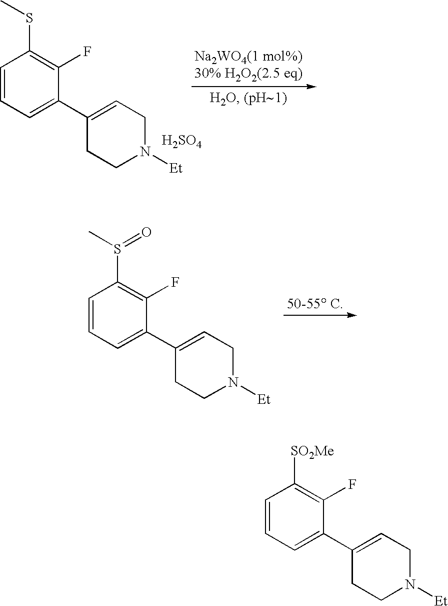

To a solution of 1-ethyl-4-[2-fluoro-3-(methylthio)phenyl]-1,2,3,6-tetrahydropyridine (22.5 g, 89.6 mmol) in sulfuric acid (1 N, 180 ml) was added sodiumtungstate dihydrate (0.29 g, 0.89 mmol), and hydrogenperoxide (30% in water, 22.9 ml, 224 mmol) was added in a rate that kept the temperature below 55° C. The mixture was stirred for 2 h and cooled to 10° C. The aqueous phase was made basic with a sodium hydroxide solution (5 M) and extracted with ethyl acetate (2×100 ml). The combined organic phase was dried (MgSO₄), concentrated, and purified by flash column chromatography (ethylacetate/methanol 1:1) to give the title compound (17.2 g). MS m/z (rel. intensity, 70 eV) 283 (M+, 63), 282 (29), 268 (bp), 146 (51), 110 (87).

### EXAMPLE 6

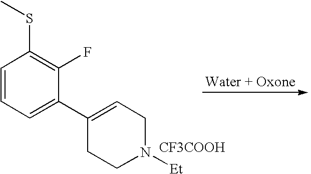

| | 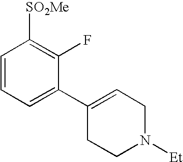 | |
|---|---|---|
| Materials | Amount | Moles |
| 1-ethyl-4-[2-fluoro-3-(methyl- | 23.5 L | 1.0 eq |
| thio)phenyl]-1,2,3,6-tetrahydropyridine | | |
| Water | 49 kg | |
| Oxone | 13.3 + | 1.1 + 0.1 + |
| | 3.6 kg | 0.1 + 0.1 eq |
| Na₂SO₃ sol (saturated) | 11 L | |
| iPrOAc | 21 L | |
| 30% NaOH | 36 L | |
| iPrOAc | 2 × 21 L | |
| NaCl sol (saturated) | 32 L | |
| iPrOAc | 12 L | |
| Silica | 20 kg | |

The solution from Example 4 was divided into two portions 23.5 L of each. The first portion was diluted with water at −3.5° C. to 7.5° C. (exothermic). Oxone was added during 90 min at −7° C. to −8.5° C. and then the reaction mixture was kept at −7° C. to 0° C. for 4.5 h and then warmed to 20° C. over a period of 120 min. The final reaction mixture was stirred at room temperature for 12 h. Oxone was then redosed 3 times at room temperature in intervals of 6-10 h. The final reaction mixture was quenched with saturated sodium sulfite solution at 0° C. The reaction solution was extracted with iPrOAc and then basified at O—C with 30% NaOH. The final water solution was extracted 2 times with iPrOAc and the combined organic phases were washed with brine. The solvents were evaporated and the final oily residue was purified with chromatography using heptane/EtOAc (1:1)+5% NEt₃ as the eluting system to give the title compound (17.2 g). MS m/z (rel. intensity, 70 eV) 283 (M+, 63), 282 (29), 268 (bp), 146 (51), 110 (87).

### EXAMPLE 7

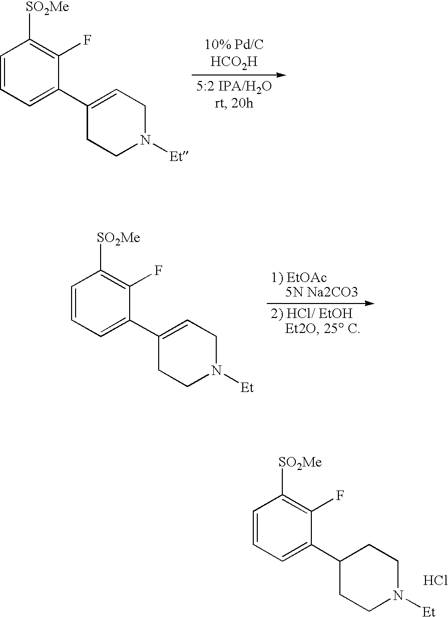

A mixture of 1-ethyl-4-[2-fluoro-3-(methylsulfonyl)phenyl]-1,2,3,6-tetrahydropyridine (5.0 g, 17.7 mmol), palladium on carbon (1.1 g) and formic acid (3.4 ml) in 2-propanol (50 ml) was hydrogenated under hydrogen at 50 psi for 15 h. The reaction mixture was filtered through a pad of celite and the filtrate was concentrated and evaporated to dryness.

Aqueous sodium carbonate (10%, 100 ml) and ethylacetate (100 ml) was added and the phases were separated. The aqueous phase was extracted with ethylacetate (2×50 ml) and the combined organic phases was dried (MgSO₄) and evaporated under reduced pressure to give an oil. Purification by flash column chromatography (ethylacetate/methanol, 1:1) gave the title compound: 2.5 g (50%). The amine was converted to the hydrochloric acid salt and recrystallized from ethanol/diethyl ether: M.p. 279-280° C. MS m/z (relative intensity, 70 eV) 285 (M+, 12), 271 (15), 270 (bp), 147 (7) 133 (8).

### EXAMPLE 8

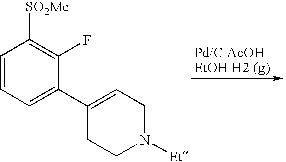

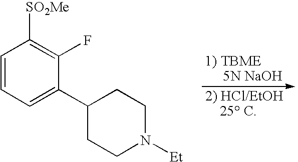

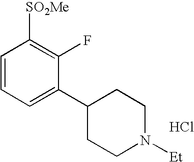

| Materials | Amount | Moles |
|---|---|---|
| 1-ethyl-4-[2-fluoro-3-(methyl- | 5.308 kg | 1.0 eq |
| thio)phenyl]-1-1,2,3,6-tetrahydropyridine | | |
| 10% Pd/C (55-57% wet, type 87L paste from JM) | 4.533 kg | |
| Ca(OAc)₂ | 296 g | 0.1 eq |
| Ethanol | 53 L | |
| Acetic Acid | 5.4 L | 5.0 eq |
| H2 | 1150 L | |
| Celite | 5.335 kg | |
| Ethanol | 10 + 26 L | |
| TBME | 53 L | |
| NaOH (5 M) | 21 L | |
| TBME | 26.4 L | |
| Sat NaCl | 21 + 21 L | |
| TBME | 5 L | |
| Silica gel | 42 kg | |
| EtOH | 90 L | |
| HCl (1.25 M in EtOH) | 6.35 L | |

The reactor was loaded with Pd/C catalyst and Ca(OAc)₂. Then the reactor was purged with nitrogen followed by addition of 1-ethyl-4-[2-fluoro-3-(methylsulfonyl)phenyl]-1,2,3,6-tetrahydropyridine, EtOH and acetic acid. The mixture was hydrogenated with hydrogen gas over a period of 12 h. The mixture was then filtered through a pad of celite which was then rinsed with EtOH. The EtOH was then evaporated and the remaining residue treated with 5 M NaOH solution. The water phase was then extracted with TBME. The combined organic phases were washed with brine and then concentrated to yield an oily residue which was purified with chromatography using heptane/EtOAc (1:1)+5% NEt₃ as the eluting system to give the title compound. The amine was then dissolved in EtOH and HCl in EtOH was added at 60° C. The final solution was slowly cooled to 20° C. and the crystallisation started. The final suspension was stirred for 1 h at 20° C. and then the crystals were filtered off and dried to yield 3.2 kg of final product. M.p. 284° C. MS m/z (relative intensity, 70 eV) 285 (M+, 12), 271 (15), 270 (bp), 147 (7) 133 (8).

### EXAMPLE 9

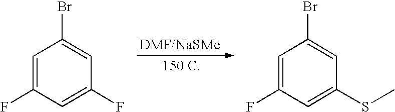

To a solution of 1-bromo-3,5-difluorobenzene (5.0 g, 25.9 mmol) in dimethylformamide (40 ml) was added sodiumthiomethylate (1.81 g, 25.9 mmol), and the mixture was heated to 150° C. for 10 min. The reaction mixture was brought to ambient temperature, quenched with saturated aqueous ammonium chloride (100 ml) and extracted with ethylacetate (3×100 ml). The combined organic phases was dried (MgSO4) and concentrated in vacuo to receive the pure title compound (3.84 g). MS m/z (rel. intensity, 70 eV) 222 (M+, 100), 220 (M+, 100), 189-(49), 187 (50), 126 (75).

### EXAMPLE 10

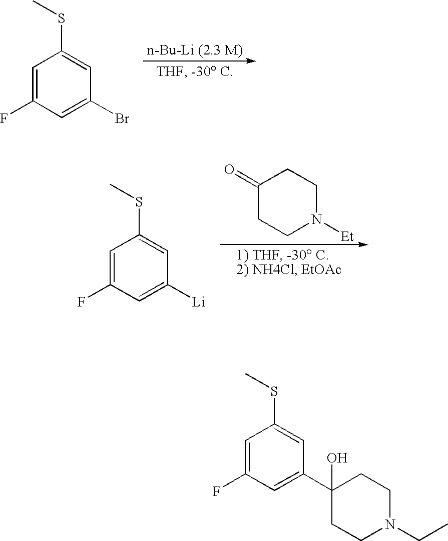

Preparation according Example 2: 1-bromo-3-fluoro-5-(methylthio)benzene (3.8 g, 17.4 mmol), dry tetrahydrofuran (70 ml), n-butyllithium (2.5 M in hexane, 7.7 ml, 19.1 mmol), 1-ethyl-4-piperidone (2.2 g, 17.4 mmol). Yield: 4.7 g. MS m/z (rel. intensity, 70 eV) 269 (M+, 73), 254 (bp), 236 (34), 109 (136), 84 (75).

### EXAMPLE 11

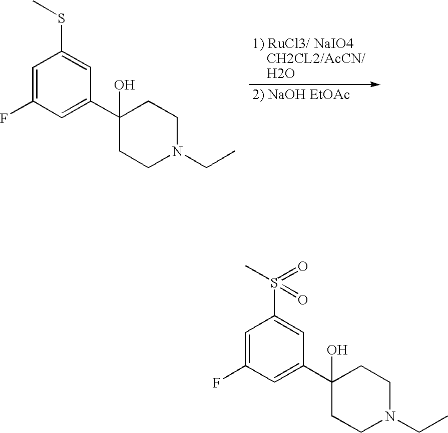

To a solution of 1-ethyl-4-[3-fluoro-5-(methylthio)phenyl]piperidin-4-ol (8.3 g, 30.7 mmol) in methylene chloride (40 ml), acetonitrile (40 ml) and water (80 ml), was added sodium periodate (19.7 g, 92.1 mmol) and ruthenium (III) chloride (15 mg, 0.05 mol %). The mixture was stirred for 0.5 h, made basic with a sodium hydroxide solution (1 M, 50 ml) and extracted with ethyl acetate (2×100 ml). The combined organic phase was dried (MgSO4) and concentrated to give the title compound (5.2 g). MS m/z (rel. intensity, 70 eV) 301 (M+, 21), 287 (16), 286 (bp), 256 (64), 84 (42).

### EXAMPLE 12

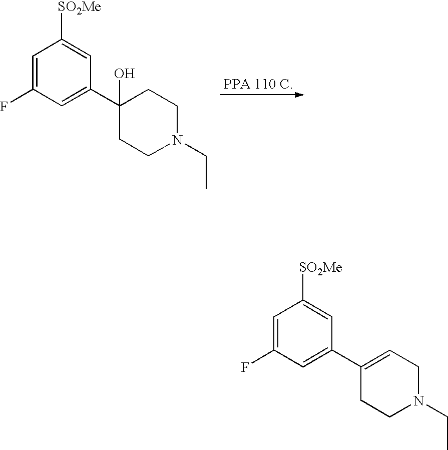

A mixture of 1-ethyl-4-[3-fluoro-5-(methylsulfonyl)phenyl]piperidin-4-ol (5.2 g, 17.3 mmol) and polyphosphoric acid (15 ml) was heated at 110° C. for 1 h. The mixture was poured on to ice and was basified with 5 M sodium hydroxide. The mixture was extracted with ethylacetate (3×100 ml) and the combined organic phases was dried (MgSO₄), filtered and evaporated to dryness to give an oil. The crude product was purified by flash column chromatography (ethylacetate/methanol 1:1) to give the title compound (2.2 g). MS m/z (rel. intensity, 70 eV) 283 (M+, 76), 282 (36), 268 (bp), 146 (18), 110 (19).

### EXAMPLE 13

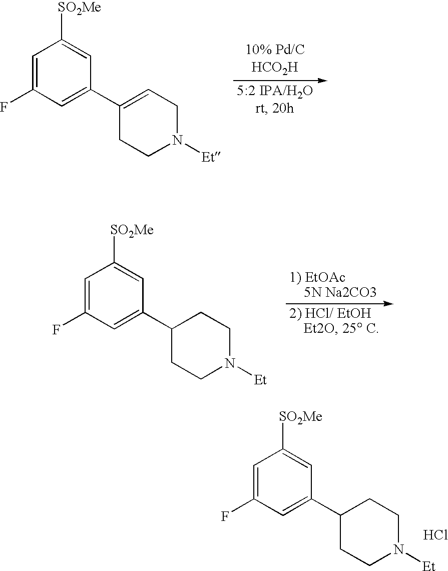

Preparation according to example 1: 1-ethyl-4-[3-fluoro-5-(methylsulfonyl)phenyl]-1,2,3,6-tetrahydropyridine (2.2 g, 7.7 mmol), palladium on carbon (0.43 g), formic acid (1.5 ml) and 2-propanol (50 ml). Yield: 1.9 g (87%). The amine was converted to the hydrochloric acid salt and recrystallized from ethanol/diethyl ether: M.p. 176-178° C. MS m/z (relative intensity, 70 eV) 285 (M⁺, 15), 284 (16), 271 (16), 270 (bp), 84 (15).

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, reaction conditions other than the particular conditions as set forth herein above may be applicable as a consequence of variations in the reagents or methodology to prepare the compounds from the processes of the invention indicated above. Likewise, the specific reactivity of starting materials may vary according to and depending upon the particular substituents present or the conditions of manufacture, and such expected variations or differences in the results are contemplated in accordance with the objects and practices of the present invention. It was intended, therefore, that the invention be defined by the scope of the claims which follow.

1. A process for preparing a compound of the formula VI: 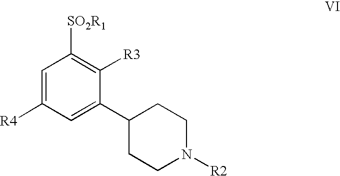
wherein:
R¹ is selected from the group consisting of:
(1) —CH₃, and
(2) —CH₂CH₃;
R² is selected from the group consisting of:
(1) —CH₂CH₃,
(2) —CH₂CH₉CH₃,
(3) —CH₂CH₂CH₂CH₃,
(4) —CH(CH₃)CH₂CH₃,
(5) —CH₂CH(CH₃)₂,
(6) —CH₂CH₂OCH₃, and
(7) —CH₂CH═CH₂;
R³ is selected from the group consisting of:
(1) hydrogen, and
(2) fluoro;
R⁴ is selected from the group consisting of:
(1) hydrogen, and
(2) fluoro;
and pharmaceutically acceptable salts thereof;
which comprises oxidizing a sulfide of the formula VIII: 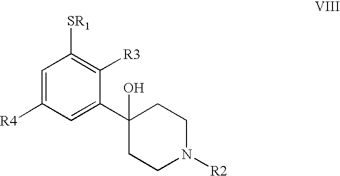
to give a compound of the formula X: 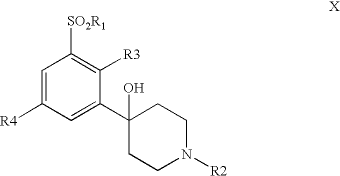
followed by dehydration of the compound X to give a compound of the formula IX 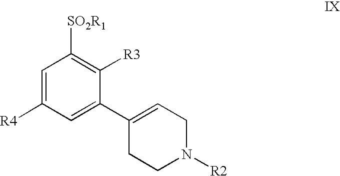
followed by catalytic reduction of the compound of formula IX to give the compound of the formula VI: 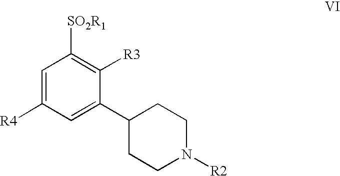
or a pharmaceutically acceptable salt thereof.

2. The process according to claim 1, in which R¹ is —CH₃, R² is —CH₂CH₃, R³ is F and R⁴ is H.

3. The process according to claim 1, in which R¹ is —CH₃; R² is —CH₂CH₃, R³ is H and R⁴ is F.

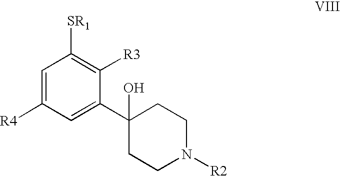

4. The process according to claim 1 wherein dehydration of the compound of formula X is conducted with a strong acid selected from sulfuric acid, hydrochloric acid, hydrofluoric acid, nitric acid and trifluoroacetic acid.

5. The process according to claim 4, wherein the dehydration of the alcohol of the formula X with a strong acid is conducted in solvent selected from toluene, xylene, hexanes and water.

6. The process according to claim 4 wherein the step of oxidizing the sulfide of the formula VII is conducted at less than 2 pH.

7. The process according claim 1, wherein oxidizing a sulfide of the formula VIII is carried out using a catalytic oxidizing agent and an oxidant.

8. The process according to claim 7, wherein the catalytic oxidizing agent is a tungsten, ruthenium, rhenium, molybdenum, osmium, silicotungstate or chromium oxidizing agent.

9. The process according to claim 8 wherein the catalytic oxidizing agent is a tungsten oxidizing agent such as e.g. sodium tungstate.

10. The process according to claim 9, wherein the oxidant is a peroxide.

11. The process according to claim 10, wherein the peroxide is sodium peroxide, hydrogen peroxide, sodium hypochlorite, sodium bromate, sodium periodate, peroxyacetic acid or peroxybenzoic acid.

12. The process according to claim 1, wherein oxidizing a sulfide of the formula VII carried out using a stoichiometric oxidant.

13. The process according to claim 12 wherein the oxidant is a peroxide, oxone, MCPBA or KMnO₄.

14. The process according to claim 1 wherein the step of oxidizing the sulfide of the formula II is conducted at a temperature between 40° C. and 60° C.

15. The process according to claim 1 wherein the catalytic reduction of the compound of the formula IX comprises catalytic hydrogenation with a palladium catalyst, a platinum catalyst or a ruthenium catalyst.

16. The process according to claim 15 wherein the catalytic reduction of the compound of the formula IX comprises catalytic hydrogenation with a palladium catalyst.

17. The process according to claim 15, wherein the catalytic reduction of the compound of the formula IX comprises catalytic hydrogenation with a palladium on carbon catalyst.

18. The process according to claim 14 wherein the catalytic reduction of the compound of the formula IX comprises catalytic hydrogenation with a 10% palladium on carbon catalyst.

19. The process according to claim 15, wherein the step of catalytic reduction of the compound of the formula IX is conducted in an aqueous solution with an alcohol.

20-24. (canceled)
